(19) United States
(12) Patent Application Publication
(10) Pub. No.: US 2018/0369485 A1
(43) Pub. Date: Dec. 27, 2018

(54) Carpule/Cartridge and Syringe Holder Device and System

(71) Applicant: Edward Yalisove, Wilmington, DE (US)

(72) Inventor: Edward Yalisove, Wilmington, DE (US)

(21) Appl. No.: 15/634,838

(22) Filed: Jun. 27, 2017

(51) Int. Cl. A61M 5/24; A61M 5/315; A61J 1/06

(52) U.S. Cl. CPC A61M 5/24; A61M 5/34; A61J 1/06; A61M 5/31566

(57) ABSTRACT

A device configured to securely hold syringes, used carpules/capsules, and unused carpules/capsules is described. The device is equipped with a first syringe holder and a second syringe holder, each disposed at differing angles with respect to a base of the device. The device is also equipped with multiple carpule/cartridge holders, each bored into the device. The entirety of the device is preferably metallic, lending to its capacity to be autoclaved for ideal sterilization before use. The device facilitates one-handed closing or capping of syringes, helping to ensure the safety of the patient and the administrator. It is envisioned that the device be used to contain liquid anesthetics to be administered to a patient. The placement of the carpule/cartridge holders enables the dentist or other dental staff to readily keep track of how much anesthetic has been applied, helping to prevent error or injury. And for documentation, to keep track of dosages used. Up to nine disposable syringes, to keep track of the quantity and type of medication injected.

### FIELD OF THE PRESENT INVENTION

The present invention relates to devices configured to assist in dental work, and more specifically relates to an apparatus configured to hold and help to keep track of carpules/cartridges used in the application and administration of anesthesia via syringes.

### BACKGROUND OF THE PRESENT INVENTION

Conventionally, local anesthetic is often used to ease the pain of dental work. The anesthetic is usually applied via a syringe, which administers the liquid anesthetic directly to the intended area(s) of the mouth.

Often, when working on more than one area of the mouth, or in cases which are known to be extremely painful, the general dentist or other authorized medical professional will administer anesthetic in multiple locations within the mouth of the patient. Sometimes, distractions can amount to mistakes, which unfortunately, can lead to injury or fatality. As such, it is critically important for the general dentist, or those authorized to administer anesthetics, to pay close attention to how much anesthetic is administered, and within what amount of time. If there were a way in which administrators could more easily keep track of used and unused carpules/cartridges, the administrators could be more confident in knowing the dosage administered, and could help to prevent overdosing or contamination.

Additionally, when using multiple carpules/cartridges of anesthetic, the administrator must change the carpule/cartridge out of the syringe. Best practice is for the needle of the syringe to be capped when not actively in use on the skin or mouth of the patient. As such, there is a desire for the syringe to be capable of being easily ‘capped’ with only one hand—namely, the same hand holding the syringe itself. Unfortunately, two-handed re-capping of syringes can be dangerous today, and it is mandated that OSHA requires single-handed recapping to avoid injury. Occasionally, administrators may accidentally prick his or her finger in the process, which can lead to infection and/or contamination. If there were a device that could assist the administrator of anesthetic during the administration process, including the ability to cap syringes with one hand, fewer injuries would result.

Similar issues also exist with cosmetic surgery applications. As with dentistry, syringes are employed, however instead of anesthetics, they conventionally contain Botox and dermal fillers. Up to nine syringes are often used during application, each of which requires a qualified medical professional keeping track of administered doses, and appropriately recapping the syringes after each use. However, disposable syringes are conventionally employed, which tend to have different diameters than that of the reusable syringes used in dentistry. If there were a device sized appropriately for cosmetic surgery applications, recapping syringes would be easier, and similarly, fewer injuries would result.

Thus, there is a need for a device configured to hold syringes, as well as used or unused carpules/cartridges such that they may be easily differentiated. Additionally, there is a need for a device that can hold syringes when not presently in use, which can facilitate recapping of the needle of the syringe without necessitating use of both hands. Such a device is preferably configured for use with any needle-based anesthetic treatments, as well as other liquids administered via syringe during conventional dentistry and medical treatments. Multiple sizes of such a device (the circumference of the syringes varies between embodiments) are preferably available to cater to both dentistry as well as cosmetic surgery applications respectively.

Presently, there are no current solutions on the market which enable a user to securely hold and re-cap two different types of anesthetic—or indeed, two syringes simultaneously, unlike the present invention. Additionally, there is not presently a solution on the market configured to hold six carpules/cartridges. While some solutions presently exist on the market to hold a single syringe, they are limited to only recapping a single syringe, and are therefore unable to hold multiple syringes. Some of these solutions are cardboard, and are therefore configured for single-use which are to be disposed after use. Conversely, the present invention is medical-grade stainless steel, and can therefore be reused and sterilized via autoclave.

### SUMMARY OF THE PRESENT INVENTION

This Summary of the Invention is provided to introduce a selection of concepts in a simplified form that are further described below in the Detailed Description. This Summary is not intended to identify key features or essential features of the claimed subject matter, nor is it intended to be used to limit the scope of the claimed subject matter.

The present invention is a holder configured for use by authorized medical personnel. Preferably, the present invention is designed to be used for the holding of at least one syringe when not in use, as well as full and/or empty anesthetic cartridges/carpules. The present invention helps medical personnel reliably track how much anesthetic has been administered, and allows for the safe and easy recapping of carpules/cartridges and/or syringes with only one hand.

As such, the present invention is the only medical device that secures various sized needle caps for safe, one-handed recapping. The present invention holds the recapped syringes during aesthetic procedures, such as neurotoxin and dermal filler injections, PRP administration, and sclerotherapy. Safe recapping and the elimination of stray needles in the workspace reduces the risk of needle stick injuries, helping to ensure the safety of both patients and the staff.

An alternate embodiment of the present invention is designed to facilitate the administration of Botox and other conventional dermal fillers also administered via syringe needle. Conventionally, disposable syringes are used in such cosmetic surgery work, and can require the use of up to nine separate syringes. As with the primary embodiment, the commonality between both embodiments is the desire for single-handed recapping of the syringe in order to prevent needle stick injuries, and to accurately keep track of the dose administered.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 exhibits a view of the present invention from the top.

FIG. 2 displays a view of the present invention as seen from the side and top.

FIG. 3 depicts a flow chart detailing the process of use of the present invention by authorized personnel.

FIG. 4 shows a flow chart detailing the process of use of an alternate embodiment of the present invention.

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s). The invention is defined by the claims appended hereto.

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to effect such feature, structure, or characteristic in connection with other embodiments whether or not explicitly described.

The present invention is a medical device configured for use by authorized medical personnel. The device of the present invention is a carpule/cartridge holder configured to facilitate one-handed capping and/or closing of syringes designed to accept such cartridges. The present invention is generally planar, preferably having a body (10) which is rectangular. The body (10) is equipped with a base (20) which is preferably level, and is configured to rest securely on a table, mobile platform, or similar medical surface. The body (10) is equipped with a first syringe holder (30) and a second syringe holder (40), each configured to securely hold a capped syringe until used, as well as after use. The bore of the first syringe holder (30) into the body (10) is preferably angled at 65 degrees. The bore of the second syringe holder (40) is preferably angled at 90 degrees.

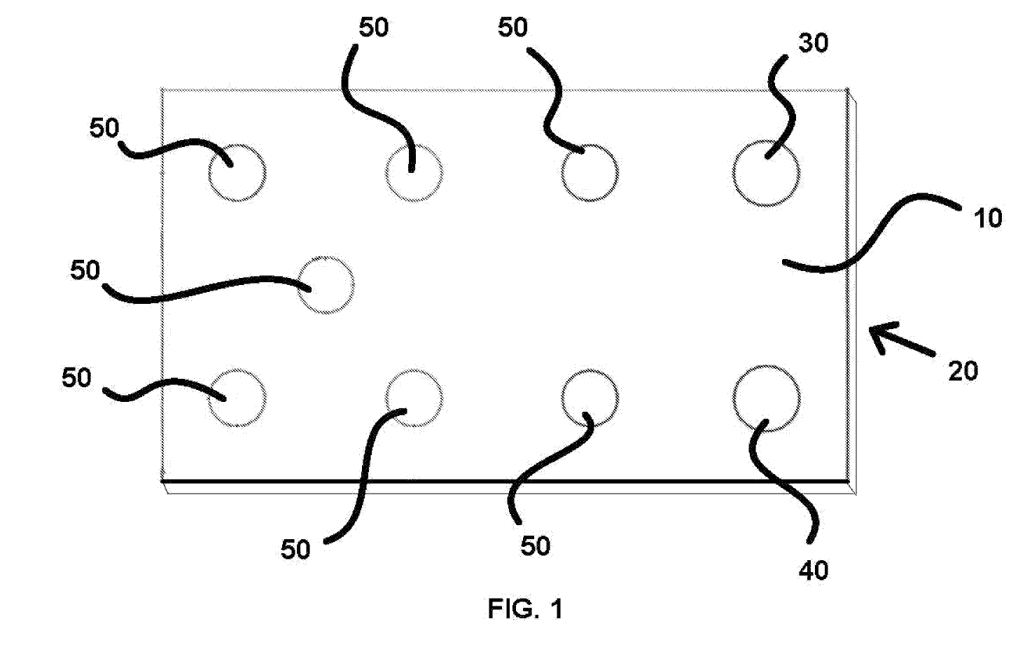

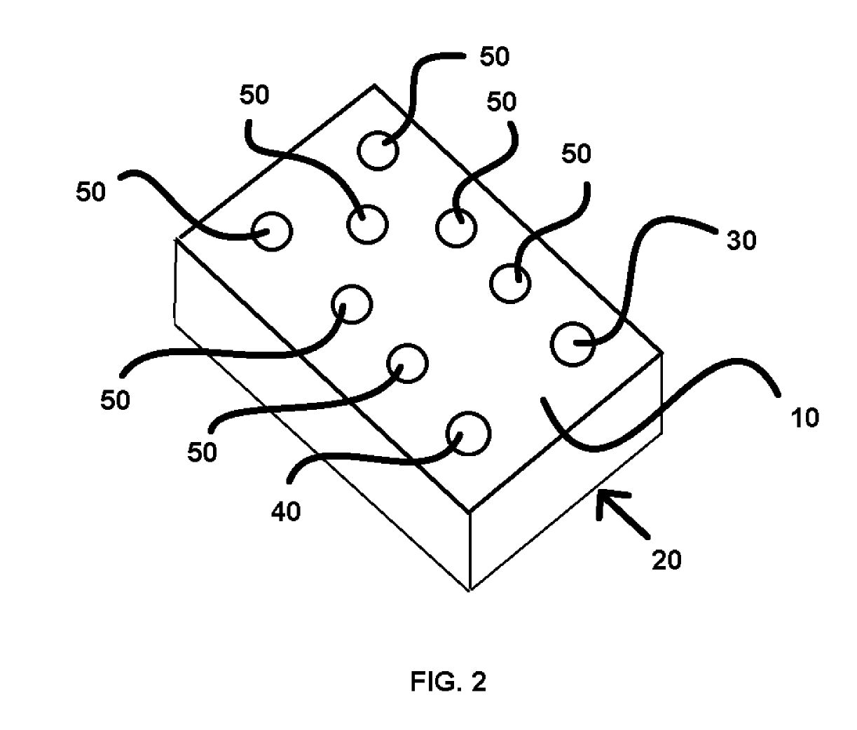

Additionally, the present invention is equipped with carpule/cartridge holders (50), which are disposed adjacent to, yet separate from the first syringe holder (30) and second syringe holder (40) as shown in FIG. 1 and FIG. 2. There are preferably six (6) carpule/cartridge holders (50) disposed on the body of the present invention, each bored into the body (10) at a 90 degree angle such that the carpule/cartridges are perpendicular to the body (10) when stowed within the carpule/cartridge holder (50) of the present invention.

The body (10) of the present invention is preferably completely made out of a single piece of metal, such as stainless steel, silver alloys, aluminum, or other similar metals. Employing metallic composition enables the entirety of the present invention to be thoroughly sterilized before each use via an autoclave.

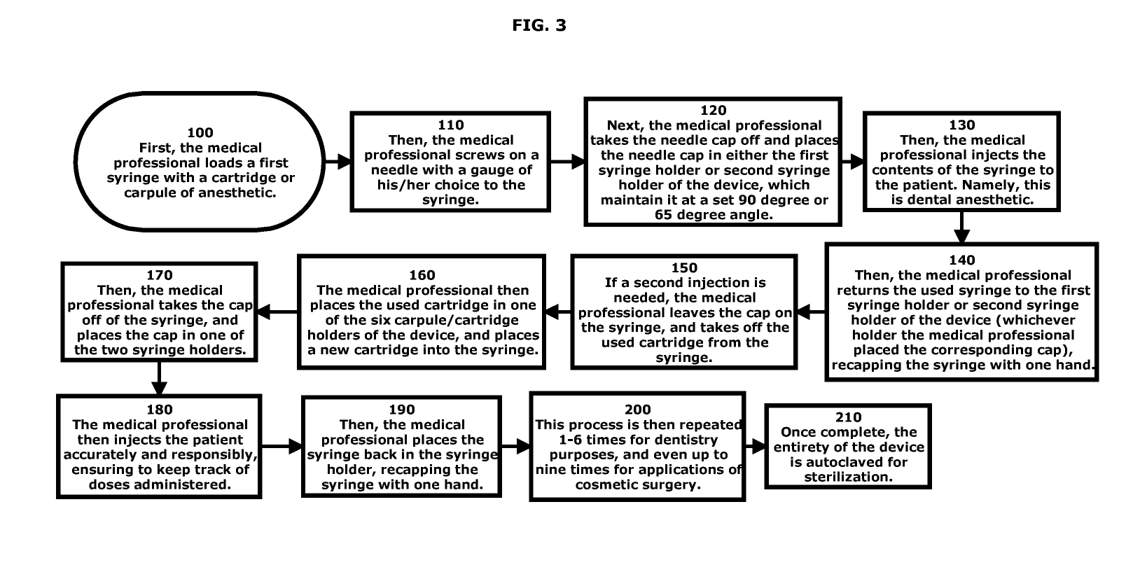

The process of installation and use of the primary embodiment of the present invention, as depicted in FIG. 3, is preferably as follows:
1. First, the medical professional loads a first syringe with a cartridge or carpule of anesthetic. (100)
2. Then, the medical professional screws on a needle with a gauge of his/her choice to the syringe. (110)
3. Next, the medical professional takes the needle cap off and places the needle cap in either the first syringe holder or second syringe holder of the device, which maintain it at a set 90 degree or 65 degree angle. (120)
4. Then, the medical professional injects the contents of the syringe to the patient. (130) Namely, this is dental anesthetic.
5. Then, the medical professional returns the used syringe to the first syringe holder or second syringe holder of the device (whichever holder the medical professional placed the corresponding cap), recapping the syringe with one hand. (140)
6. If a second injection is needed, the medical professional leaves the cap on the syringe, and takes off the used cartridge from the syringe. (150)
7. The medical professional then places the used cartridge in one of the six carpule/cartridge holders of the device, and places a new cartridge into the syringe. (160)
8. Then, the medical professional takes the cap off of the syringe, and places the cap in one of the two syringe holders. (170)
9. The medical professional then injects the patient accurately and responsibly, ensuring to keep track of doses administered. (180)
10. Then, the medical professional places the syringe back in the syringe holder, recapping the syringe with one hand. (190)
11. This process is then repeated 1-6 times for dentistry purposes, and even up to nine times for applications of cosmetic surgery. (200)
12. Once complete, the entirety of the device is autoclaved for sterilization. (210)

An alternate embodiment of the present invention is designed to facilitate the administration of Botox and conventional dermal fillers also administered via syringe needle. Conventionally, disposable syringes are used in such cosmetic surgery work, and can require the use of up to nine separate syringes. Both embodiments allow for single-handed recapping of syringes in order to prevent needle stick injuries, and to accurately keep track of the dose administered. As such, the second, or alternate embodiment of the present invention is also equipped with carpule/cartridge holders (50), as shown, in addition to the first syringe holder (30) and second syringe holder (40) of the body (10). In this alternate embodiment of the present invention, both the first syringe holder (30) and the second syringe holder (40) are disposed at a 90 degree angle with respect to the base (20), unlike the primary first embodiment, which maintains the second syringe holder (40) at 65 degrees.

It should be understood that, in use of the second embodiment of the present invention, the carpule/cartridge holders (50) are configured to hold disposable syringes, especially after use, to facilitate tracking of doses administered (as disposable syringes do not employ cartridges or carpules).

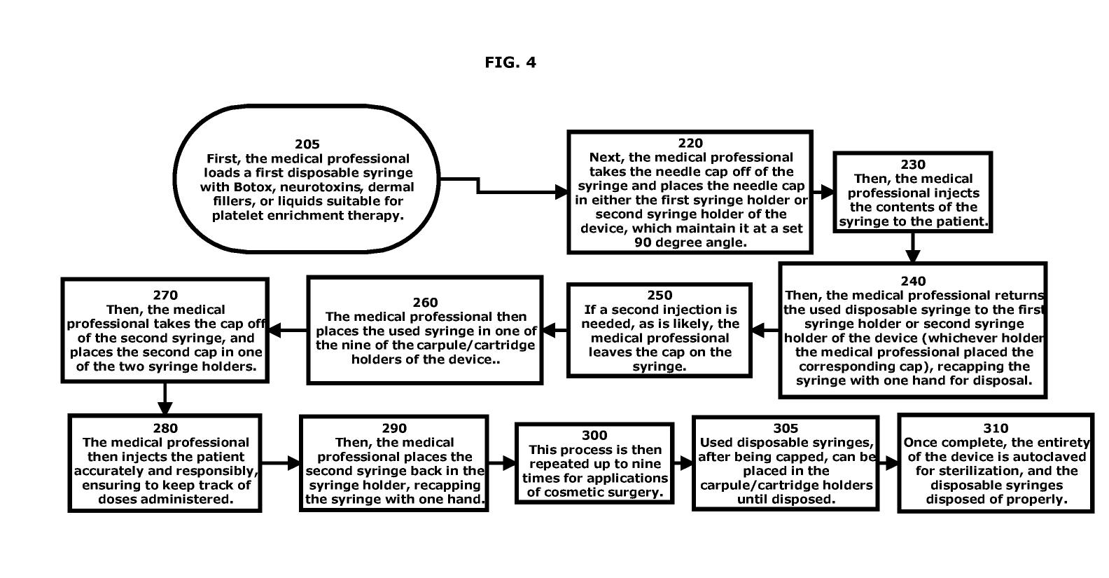

The method of use of the alternate embodiment of the present invention, used for surgical applications as shown in FIG. 4, is preferably as follows:
1. First, the medical professional loads a first disposable syringe with Botox, neurotoxins, dermal fillers, or liquids suitable for platelet enrichment therapy. (205)
2. Next, the medical professional takes the needle cap off and places the needle cap in either the first syringe holder or second syringe holder of the device, which maintain it at a set 90 degree angle. (220)
3. Then, the medical professional injects the contents of the syringe to the patient. (230) Namely, this is Botox, dermal fillers, and other cosmetic surgery, needle-based injections.
4. Then, the medical professional returns the used disposable syringe to the first syringe holder or second syringe holder of the device (whichever holder the medical professional placed the corresponding cap), recapping the syringe with one hand for disposal. (240)
5. If a second injection is needed, the medical professional leaves the cap on the syringe. (250)
6. The medical professional then places the used cartridge in one of the six carpule/cartridge holders of the device, and places a new cartridge into the syringe. (260)
7. Then, the medical professional takes the cap off of the syringe, and places the cap in one of the two syringe holders. (270)
8. The medical professional then injects the patient accurately and responsibly, ensuring to keep track of doses administered. (280)
9. Then, the medical professional places the syringe back in the syringe holder, recapping the syringe with one hand. (290)
10. This process is then repeated up to nine times for applications of cosmetic surgery. (300)
11. Used disposable syringes, after being capped, can be placed in the carpule/cartridge holders to facilitate tracking of doses administered until disposed. (305)
12. Once complete, the entirety of the device is autoclaved for sterilization, and the disposable syringes disposed of properly. (310)

It should be understood that the first syringe holder (30) and the second syringe holder (40) may be made available in a variety of circumferences to match predominant needle sizes, as well as common proprietary cap designs of conventional syringes. It is known that larger needles, and often therefore larger caps are present with the application of Botox, yet dermal fillers are smaller. Thus, it is advantageous to have both the first syringe holder (30) and the second syringe holder (40) which vary in size in accordance with their trade application.

It should also be understood that both embodiments of the present invention may be autoclaved, for sterilization, and are preferably made of medical-grade stainless steel.

It should similarly be understood that the first embodiment of the present invention, envisioned for use in a dental setting, are equipped with six (6) carpule/cartridge holders (50), however it is envisioned that more or less carpule/cartridge holders (50). The second embodiment may be equipped with up to nine carpule/cartridge holders (50), however they are configured to hold used disposable syringes to track doses administered. It should be understood that this second embodiment may be equipped with fewer, or more than nine carpule/cartridge holders (50) in some embodiments of the present invention.

Additionally, some alternate embodiments of the present invention may be configured to be disposable, and as such, are preferably fashioned out of a durable plastic or plastic composite material. In such plastic embodiments, the base (20) of the present invention is larger than that of the stainless steel embodiments to compensate for the lighter weight, helping to ensure that the present invention does not tip over during use. The base (20) is larger in order to have a greater surface area in contact with the surface on which the present invention is placed.

It should be understood that the stainless steel composition of the preferred embodiment of the present invention helps to ensure that the present invention is equipped with enough weight to prevent accidental tipping. Without this weight, the present invention may tip over. In plastic, disposable embodiments of the present invention, the base (20) is larger, ensuring that this embodiment is not prone to tipping, while remaining disposable.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A medical device comprising:
a body, said body having a base disposed at a bottom of said body;
wherein said body is generally planar;
a first syringe holder, said first syringe holder disposed within said body;
a second syringe holder, said second syringe holder disposed within said body, adjacent to said first syringe holder;
carpule/cartridge holders, said carpule/cartridge holders disposed within said body;
wherein said carpule/cartridge holders are bored into said body; and
wherein said body is unitary.

2. The device of claim 1, wherein said first syringe holder is disposed at a 90 degree angle with respect to said base.

3. The device of claim 1, wherein said second syringe holder is disposed at a 65 degree angle with respect to said base.

4. The device of claim 1, wherein said second syringe holder is disposed at a 90 degree angle with respect to said base.

5. The device of claim 1, wherein said first syringe holder and said second syringe holder are configured to hold a first syringe having a first cap and a second syringe having a second cap respectfully.

6. The device of claim 5, wherein said first syringe holder is configured to hold said first cap of said first syringe at an angle, when said first syringe is in active use, that facilitates one-handed recapping of said first syringe after use of said first syringe.

7. The device of claim 1, wherein said body is made of surgical-grade stainless steel.

8. The device of claim 2, wherein said second syringe holder is disposed at a 65 degree angle with respect to said base.

9. The device of claim 2, wherein said first syringe holder and said second syringe holder are configured to hold a first syringe having a first cap and a second syringe having a second cap respectfully.

10. A method of onehandedly administering anesthetic via a syringe equipped with a cap comprising:
loading the syringe with a cartridge/carpule of anesthetic;
affixing a needle of the appropriate gauge to the syringe;
removing the cap of the syringe, revealing the needle;
placing the cap into a first syringe holder disposed on a metallic body;
wherein the cap rests within the first syringe holder such that a top of the cap is directed towards a base of the metallic body;
administering the anesthetic to a patient;
returning the syringe to the first syringe holder, recapping the syringe in one motion with one hand;
removing the cartridge/carpule;
placing the cartridge/carpule into a cartridge/carpule holder; and
wherein the cartridge/carpule holder is bored into the metallic body.

11. The method of claim 10, further comprising:
the patient having a need for additional anesthetic;
loading the syringe with a second cartridge/carpule;
removing the cap from the syringe;
placing the cap within the first syringe holder such that the top of the cap is oriented towards the base of the metallic body;
administering the additional anesthetic;
returning the syringe to the first syringe holder, recapping the syringe with a single motion onehandedly;
removing the second cartridge/carpule from the syringe; and
placing the second cartridge/carpule into a cartridge/carpule holder.

12. The method of claim 11, wherein six cartridge/carpule holders are present within the metallic body, each configured to hold a cartridge/carpule prior or post-use, at a 90 degree angle.

13. A medical device comprising:
a body, said body having a base disposed at a bottom of said body;
wherein said body is generally planar;
wherein said base is wider than said body;
wherein said base and said body are composed of a plastic polymer;
wherein said base and said body are permanently connected;
a first syringe holder, said first syringe holder disposed within said body;
a second syringe holder, said second syringe holder disposed within said body, adjacent to said first syringe holder;
carpule/cartridge holders, said carpule/cartridge holders disposed within said body;
wherein said carpule/cartridge holders are bored into said body;
wherein said body is unitary; and
wherein said body and said base are disposable.
